SEQUENCE LISTING (2 sequences)

SEQ ID NO: 1 - DNA, 20 bp, Artificial Sequence
  Synthetic primer for CD2F1 mouse
aatggagactgatgcgctct

SEQ ID NO: 2 - DNA, 20 bp, Artificial Sequence
  Synthetic primer for CD2F1 mouse
actcttgccacacaggctct

(19) United States
(12) Patent Application Publication
(10) Pub. No.: US 2019/0070139 A1
(43) Pub. Date: Mar. 7, 2019

(54) AGENT FOR IMPROVING PHYSICAL FITNESS

(71) Applicant: AJINOMOTO CO., INC, Tokyo (JP)

(72) Inventors: Yoshihito NOGUSA, Kawasaki-shi (JP); Ami MIZUGAKI, Kawasaki-shi (JP)

(73) Assignee: AJINOMOTO CO., INC., Tokyo (JP)

(21) Appl. No.: 16/130,500

(22) Filed: Sep. 13, 2018

Related U.S. Application Data
(63) Continuation of application No. PCT/JP2017/010461, filed on Mar. 15, 2017.

(30) Foreign Application Priority Data
Mar. 16, 2016 (JP) 2016-053100

(51) Int. Cl. A61K 31/198; A23L 33/175; A61P 43/00; A61P 21/06

(52) U.S. Cl. CPC A61K 31/198; A23L 33/175; A23V 2002/00; A61P 21/06; A61P 43/00

(57) ABSTRACT

Ingestion or administration of cysteine is effective for improving physical capacity for activity and provides a superior improving effect as compared to conventional compositions for the enhancement of improvement of physical capacity for activity relating to physical functions.

### CROSS REFERENCES TO RELATED APPLICATIONS

This application is a continuation of International Patent Application No. PCT/JP2017/010461, filed on Mar. 15, 2017, and claims priority to Japanese Patent Application No. 2016-053100, filed on Mar. 16, 2016, both of which are incorporated herein by reference in their entireties.

### BACKGROUND OF THE INVENTION

### Field of the Invention

The present invention relates to agents for improving physical capacity for activity. The present invention also relates to methods for improving physical capacity for activity.

### Discussion of the Background

Exercise, which improves physical functions, is often important for athletes in improving athletic performance, and important for people with diseases such as lifestyle-related disease and the like and the elderly people in treating the disease and preventing aging. Various physical functions are improved by exercise and, for example, improvements of physical capacity for activity such as enhanced muscular strength, improved endurance capacity and the like can be mentioned. Exercise for improving such physical functions is called training. However, training for improving the physical function cannot achieve a sufficient effect in a short period of time, generally requires repeats of long-term exercise, and is difficult to continue mentally and physically for patients with diseases. Also for top-level athletes, the amount and intensity of exercise for improving physical function become high, thus leading to problems such as sluggish improvement in exercise capacity and risk of injury.

Therefore, it is very important for patients undergoing exercise therapy and top-level athletes to efficiently improve their physical capacity for activity by training.

To improve physical capacity for activity of athletes, it is desirable from the aspect of prevention of doping to ingest nutrients generally used for foods. In addition, it is highly likely that patients have already been taking pharmaceutical products. Thus, it is also desirable to improve physical capacity for activity of patients by ingestion of nutrients.

In training athletes requiring endurance capacity, glycogen loading is known as a method for improving physical capacity for activity by ingestion of nutrients. Increase in the amount of glycogen in the muscle is known to improve exercise capacity. Glycogen loading includes ingesting low-carbohydrate diet and performing intensive exercise to drastically reduce the amount of glycogen in the muscle, and subsequently switching to a high-carbohydrate diet to more effectively increase glycogen in the muscle. This is a method for improving physical capacity for activity by ingesting nutrients and is considered to be desirable. However, it requires long-term implementation, and complicated processes such as preparation of a meal with calculated nutritional values, setting of an exercise program to deplete glycogen and the like.

As an example of improved exercise capacity by ingestion of nutrients, JP-A-2008-255033 and WO 2008/105368, both of which are incorporated herein by reference in their entireties, report that amino acid mixture improved endurance capacity. Since amino acid is a nutrient naturally contained in foods, an amino acid mixture is desirable as a means. The aforementioned patent documents show the effects of a mixture containing plural amino acids; however, they do not suggest the effect of cystine.

Murakami S. et al., J. Int. Soc. Sports Nutr. 7(1):23 (2010) and Murakami S. et al., Biosci. Biotechnol. Biochem. 73(4):817-821 (2009), both of which are incorporated herein by reference in their entireties, report that ingestion of cystine and theanine for long-distance runner suppressed impairment of immune function and muscle disorder due to skeletal muscle damage after strenuous endurance exercise.

However, Murakami S. et al., J. Int. Soc. Sports Nutr. 7(1):23 (2010) and Murakami S. et al., Biosci. Biotechnol. Biochem. 73(4):817-821 (2009) do not refer to the effect of cystine and theanine on the exercise capacity and physical capacity for activity.

### SUMMARY OF THE INVENTION

Accordingly, it is one object of the present invention to provide novel nutritional compositions capable of providing a superior improving effect as compared to conventional compositions on the enhancement of physical capacity for activity relating to physical functions.

It is another object of the present invention to provide novel methods for improving physical capacity for activity relating to physical functions.

These and other objects, which will become apparent during the following detailed description, have been achieved by the inventors' discovery that cystine further enhances an increasing effect on the amount of glycogen in the muscle or an improving effect on the physical capacity for activity by exercise (training). Based on such finding, they have conducted further studies and completed the present invention.

Accordingly, the present invention provides the following:

(1) An agent for improving physical capacity for activity, comprising cystine as an active ingredient.

(2) The agent of (1], wherein the agent improves an ability to continue physical activity.

(3) The agent of (1) or (2), wherein the content of cystine is 2.5 mg to 15 g as an intake or dose per one time.

(4) The agent of any one of (1) to (3), wherein the content of cystine is not less than 1 wt % relative to the total amino acid content.

(5) The agent of any one of (1) to (4), further comprising at least one amino acid selected from the group consisting of glutamine, serine, histidine, arginine, valine, leucine, isoleucine, alanine and proline.

(6) The agent of (5), wherein a total of the content of cystine and the content of at least one amino acid selected from the group consisting of glutamine, serine, histidine, arginine, valine, leucine, isoleucine, alanine and proline is not less than 1.1 wt % relative to the total amino acid content.

(7) The agent of (5) or (6), wherein a weight ratio of the content of cystine (a) and the content of at least one amino acid selected from the group consisting of glutamine, serine, histidine, arginine, valine, leucine, isoleucine, alanine and proline (b) ((a):(b)) is 1:0.01 to 100.

(8) The agent of any one of (1) to (7), wherein the agent is orally ingested or orally administered.

(9) The agent of any one of (1) to (8), wherein the agent is ingested or administered at least once before start of exercise.

(10) The agent of any one of (1) to (9), wherein the agent is ingested or administered at least once during exercise or after completion of exercise.

(11) The agent of any one of (1) to (10), wherein the content of cystine is the highest or the second highest of all the amino acids contained.

(12) The agent of any one of (1) to (11), wherein the agent is an agent for improving muscular endurance capacity or an agent for improving whole body endurance capacity.

(13) The agent of any one of (1) to (11), wherein the agent is an agent for increasing the amount of glycogen in muscle.

(14) The agent of (13), wherein the agent is an agent for enhancing glycogen loading.

(15) A medicament comprising the agent of any one of (1) to (14).

(16) A food composition comprising the agent of any one of (1) to (14).

(17) A commercial package comprising the agent of any one of (1) to (14), and a written matter stating that the agent can or should be used for improving physical capacity for activity.

(18) A method for improving physical capacity for activity, comprising ingestion or administration of cystine in an amount effective for improving physical ability for physical activity to a subject animal in need of improvement of physical capacity for activity.

(19) The method of (18), wherein the method improves the ability to continue physical activity.

(20) The method of (18) or (19), wherein an intake or dose per one time of the cystine is 2.5 mg to 15 g.

(21) The method of any one of (18) to (20), further comprising ingestion or administration of at least one amino acid selected from the group consisting of glutamine, serine, histidine, arginine, valine, leucine, isoleucine, alanine and proline in an amount effective for improving physical capacity for activity.

(22) The method of (21), wherein an intake or dose per one time of the at least one amino acid selected from the group consisting of glutamine, serine, histidine, arginine, valine, leucine, isoleucine, alanine and proline is 10 mg to 10 g.

(23) The method of (21) or (22), wherein a weight ratio of the intake or dose per one time of cystine (c) and the intake or dose per one time of at least one amino acid selected from the group consisting of glutamine, serine, histidine, arginine, valine, leucine, isoleucine, alanine and proline (d) ((c):(d)) is 1:0.01 to 100.

(24) The method of any one of (18) to (23), comprising oral ingestion or oral administration.

(25) The method of any one of (18) to (24), comprising ingestion or administration at least once before start of exercise.

(26) The method of any one of (18) to (25), comprising ingestion or administration at least once during exercise or after completion of exercise.

(27) The method of any one of (18) to (26), wherein the intake or dose of cystine is the highest or the second highest in the intakes or doses of all the amino acids.

(28) The method of any one of (18) to (27), wherein the method improves physical capacity for activity during exercise therapy.

### Effect of the Invention

The agent for improving physical capacity for activity of the present invention improves physical capacity for activity, particularly ability to continue physical activity such as muscular endurance capacity and whole body endurance capacity. Also, the agent for improving physical capacity for activity of the present invention increases the amount of glycogen in muscle.

Therefore, the agent for improving physical capacity for activity of the present invention is effectively used for increasing the amount of glycogen in muscle or improving the ability to continue physical activity (hereinafter to be also referred to as “endurance capacity” in the present specification) and, particularly, can further enhance the effect of increasing the amount of glycogen in muscle by exercise (training), and the effect of improving muscular endurance capacity and whole body endurance capacity.

In addition, the agent for improving physical capacity for activity of the present invention contains amino acids rich in food experience such as cystine and the like as the active ingredient. Therefore, the agent has high safety, hardly causes side effects, and is extremely advantageous.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 is a schematic view showing the evaluation method of Experimental Example 1.

FIG. 2 is a graph showing the effect of cystine on improving endurance capacity in Experimental Example 1. In the Figure, “Ex” shows a group which was given a normal diet and exercised, and “Ex+Cyt” shows a group which was given a diet containing 2 wt % cystine and exercised. The numerical values on the vertical axis show the running time (min) to exhaustion. “*” and “**” respectively mean significant at P<0.05 and P<0.01.

FIG. 3 is a graph showing an effect of cystine on increasing the amount of glycogen in gastrocnemius muscle in Experimental Example 1. In the graph, the symbols “a”, “b”, “c” mean significant at P<0.05 between different alphabets. In the Figure, “Sed” shows a group which was given a normal diet and was not exercised, “Ex” shows a group which was given a normal diet and exercised for 3 consecutive days, and “Ex+Cyt” shows a group which was given a diet containing 2 wt % cystine and exercised for 3 consecutive days.

FIG. 4 is a graph showing the effect of cystine ingested after exercise on improving endurance capacity (the difference in endurance capacity between day 1 and day 2) in Experimental Example 2. In the Figure, “Ex” shows a group which was given a normal diet and exercised to exhaustion for 2 consecutive days, and “Ex+Cyt” shows a group which was given a diet containing 2 wt % cystine after exercise on day 1 and exercised on day 2 as well.

FIG. 5 is a graph showing the effect of the agent for improving physical capacity for activity of Example 1 of the present invention on improving endurance capacity in Experimental Example 3. In the Figure, “1d” is day 1 of exercise, “2d” is day 2 of exercise, “Ex-Con” shows a group which was given a normal diet containing the amino acid composition of Comparative Example and exercised to exhaustion for 2 consecutive days, and “Ex-Cyt” shows a group which was given a normal diet containing the agent for improving physical capacity for activity of Example 1 and exercised to exhaustion for 2 consecutive days. “**” means significant at P<0.01.

FIG. 6 is a graph showing the difference in endurance capacity between day 1 and day 2 in Experimental Example 3. In the Figure, “Ex-Con” shows a group which was given a normal diet containing the amino acid composition of Comparative Example and exercised to exhaustion for 2 consecutive days, and “Ex-Cyt” shows a group which was given a normal diet containing the agent for improving physical capacity for activity of Example 1 and exercised to exhaustion for 2 consecutive days. “**” means significant at P<0.01.

FIG. 7 is a graph showing the effect of the agent for improving physical capacity for activity of Example 1 of the present invention on the amount of glycogen in gastrocnemius muscle in Experimental Example 3. In the Figure, “Sed-Con” shows a group which was given a normal diet containing the amino acid composition of Comparative Example and was not exercised, “Ex-Con” shows a group which was given a normal diet containing the amino acid composition of Comparative Example and exercised to exhaustion for 2 consecutive days, “Sed-Cyt” shows a group which was given a normal diet containing the agent for improving physical capacity for activity of Example 1 and was not exercised, and “Ex-Cyt” shows a group which was given a normal diet containing the agent for improving physical capacity for activity of Example 1 and exercised to exhaustion for 2 consecutive days. “*” and “**” respectively mean significant at P<0.05 and P<0.01.

FIG. 8 is a graph showing the effect of the agent for improving physical capacity for activity of Example 1 of the present invention on the expression of glucose transporter 4 gene in gastrocnemius muscle in Experimental Example 3. In the Figure, “Sed-Con” shows a group which was given a normal diet containing the amino acid composition of Comparative Example and was not exercised, “Ex-Con” shows a group which was given a normal diet containing the amino acid composition of Comparative Example and exercised to exhaustion for 2 consecutive days, “Sed-Cyt” shows a group which was given a normal diet containing the agent for improving physical capacity for activity of Example 1 and was not exercised, and “Ex-Cyt” shows a group which was given a normal diet containing the agent for improving physical capacity for activity of Example 1 and exercised to exhaustion for 2 consecutive days. In the graph, the symbols “a”, “b”, “c”, “d” mean significant at P<0.05 between different alphabets.

FIG. 9 is a graph showing the effect of the agent for improving physical capacity for activity of Example 2 of the present invention on improving endurance capacity in Experimental Example 4. In the Figure, “Ex-Con” shows a group which was given a normal diet and exercised to exhaustion for 2 consecutive days, and “Ex-CG” shows a group which was given a normal diet containing the agent for improving physical capacity for activity of Example 2 and exercised to exhaustion for 2 consecutive days. “*” means significant at P<0.05.

FIG. 10 is a graph showing the difference in endurance capacity between day 1 and day 2 in Experimental Example 4. In the Figure, “Ex-Con” shows a group which was given a normal diet and exercised to exhaustion for 2 consecutive days, and “Ex-CG” shows a group which was given a normal diet containing the agent for improving physical capacity for activity of Example 2 and exercised to exhaustion for 2 consecutive days. “*” means significant at P<0.05.

FIG. 11 is a graph showing the effect of the agent for improving physical capacity for activity of Example 2 of the present invention on the amount of glycogen in gastrocnemius muscle in Experimental Example 4. In the Figure, “Ex-Con” shows a group which was given a normal diet and exercised to exhaustion for 2 consecutive days, and “Ex-CG” shows a group which was given a normal diet containing the agent for improving physical capacity for activity of Example 2 and exercised to exhaustion for 2 consecutive days. “*” means significant at P<0.05.

### DETAILED DESCRIPTION OF THE PREFERRED EMBODIMENTS

The agent for improving physical capacity for activity of the present invention (hereinafter to be also referred to as “the agent of the present invention”), contains cystine as the active ingredient.

In the present specification, the term “physical capacity for activity” refers to one of the two into which the “physical capacity” is largely divided, that is, physical abilities that provide a basis for physical activity.

The “physical capacity” is divided into physical capacity for activity, that is, physical ability to actually move the body and act, and physical capacity for defense referring to immunity and resistance to disease and stress, and the ability to adapt to the environment. The “physical capacity for activity” is divided into ability to take action such as muscular strength, instantaneous force and the like, ability to continue performance such as muscular endurance capacity, whole body endurance capacity and the like, and ability to adjust behavior such as agility, balance, sophistication, flexibility.

The agent for improving physical capacity for activity of the present invention mainly improves the ability to continue exercise performance, that is, improves muscular endurance capacity and whole body endurance capacity.

Here, the “muscular endurance capacity” refers to the ability of a muscle to continuously repeat contraction, that is, the ability to continue exercising using a part of the body muscle for a long time, and the “whole body endurance capacity” refers to the ability necessary to continue exercise using the muscle of the whole body for a long time.

In addition, the agent for improving physical capacity for activity of the present invention increases the amount of glycogen in muscle. Therefore, the agent for improving physical capacity for activity of the present invention promotes glycogen loading and can increase the limit of exercise capacity.

Furthermore, the agent for improving physical capacity for activity of the present invention further enhances the effect of increasing the amount of glycogen in muscle by exercise (training), or the effect of improving muscular endurance capacity or whole body endurance capacity.

Cystine, namely, 3,3′-dithiobis(2-aminopropanoic acid), contained in the agent of the present invention as an active ingredient can be used in any of L-form, D-form and DL-form. It is preferably L-form or DL-form, further preferably L-form.

Proteins and peptides containing cystine as a constituent amino acid can be used as a cystine source. Since digestion is not required, free cystine is preferably used.

Cystine can be used not only in a free form but also a salt form. The term “cystine” in the present specification is a concept encompassing even a salt. As the salt form, acid addition salt, salt with base and the like can be mentioned, and a pharmacologically acceptable salt is preferably selected.

Concrete examples of the cystine salt include salts with inorganic bases, salts with organic bases, salts with inorganic acids, salts with organic acids, salts with amino acid and the like.

Examples of the salts with inorganic bases include salts with alkali metals such as lithium, sodium, potassium and the like, salts with alkaline earth metals such as magnesium, calcium and the like, ammonium salt and the like.

Examples of the salts with organic bases include salts with alkanolamine such as monoethanolamine, diethanolamine, triethanolamine and the like, salts with heterocyclic amine morpholine, piperidine and the like, and the like.

Examples of the salts with inorganic acids include salts with hydrohalic acid (hydrochloric acid, hydrobromic acid, hydroiodic acid etc.), sulfuric acid, nitric acid, phosphoric acid and the like.

Examples of the salts with organic acids include salts with monocarboxylic acid such as formic acid, acetic acid, propanoic acid and the like; salts with saturated dicarboxylic acid such as oxalic acid, malonic acid, malic acid, succinic acid and the like; salts with unsaturated dicarboxylic acid such as maleic acid, fumaric acid and the like; salts with tricarboxylic acid such as citric acid and the like; salts with keto acid such as α-ketoglutaric acid and the like.

Examples of the salts with amino acid include salts with aliphatic amino acid such as glycine, alanine and the like; salts with aromatic amino acid such as phenylalanine and the like; salts with basic amino acid such as lysine and the like; salts with acidic amino acid such as aspartic acid, glutamic acid and the like; salts with amino acid forming lactam such as pyroglutamic acid and the like; and the like.

In the present invention, one kind of cystine in the above-mentioned free form or a salt form may be used singly, or two or more kinds thereof may be used in combination.

Cystine in a free form and hydrochloride and the like are preferable, and cystine in a free form is more preferably used for the object of the present invention.

In the present invention, cystine in a free form or in the form of a salt to be used may be extracted from animals, plants or the like, which are naturally present, and purified, or obtained by a chemical synthesis method, a fermentation method, an enzyme method or a gene recombinant method. Commercially available products provided by each company may also be utilized.

The content of cystine in the agent of the present invention is preferably not less than 2.5 mg, more preferably not less than 6 mg, further preferably not less than 10 mg, particularly preferably not less than 70 mg, as intake or dose per one time by human, from the aspect of an improvement effect on the physical capacity for activity. From the aspect of convenience for ingestion or administration, the content of cystine in the agent of the present invention is preferably not more than 15 g, more preferably not more than 12 g, further preferably not more than 10 g, as intake or dose per one time by human.

In the present specification, the content of amino acid such as cystine in the agent of the present invention when it is contained in the form of a salt is shown by the content converted to that of a free form.

Furthermore, from the aspect of an effect on improving the physical capacity for activity, the content of cystine in the agent of the present invention is preferably not less than 1 wt %, more preferably not less than 3 wt %, more preferably not less than 10 wt %, particularly preferably not less than 10 wt %, relative to the total amino acid content. In addition, the content of cystine in the agent of the present invention is more preferably not more than 90 wt %, particularly preferably not more than 80 wt %, relative to the total amino acid content.

The content of cystine is preferably the highest or second highest in all amino acids contained in the agent of the present invention. When the content of cystine is higher than other amino acids in the agent of the present invention, an efficient function as an active ingredient for improving physical capacity for activity can be exhibited.

The agent of the present invention may further contain, as other amino acid in addition to cystine, at least one amino acid selected from the group consisting of glutamine, serine, histidine, arginine, valine, leucine, isoleucine, alanine and praline. The further presence of the aforementioned at least one amino acid in addition to cystine enhances the effect of the agent of the present invention to improve physical capacity for activity. It is preferable for the agent of the present invention to contain, as other amino acid in addition to cystine, at least one amino acid selected from the group consisting of leucine, isoleucine, valine, glutamine, alanine and praline.

Particularly, in one embodiment of the present invention, it is preferable to contain, as other amino acid in addition to cystine, at least one amino acid selected from the group consisting of leucine, isoleucine and valine, further preferably leucine, isoleucine and valine.

In another embodiment, it is more preferable to contain glutamine as other amino acid in addition to cystine.

In still another embodiment, it is more preferable to contain at least one amino acid selected from the group consisting of alanine and proline as other amino acid in addition to cystine.

In the present invention, as glutamine, serine, histidine, arginine, valine, leucine, isoleucine, alanine and praline, any of L-form, D-form and DL-form thereof may be used. Preferred is L-form or DL-form, and more preferred is L-form.

These amino acids can be used not only in a free form but also in the form of a salt. The terms “glutamine”, “serine”, “histidine”, “arginine”, “valine”, “leucine”, “isoleucine”, “alanine” and “praline” in the present specification are concepts including salts.

As the form of a salt, an acid addition salt, a salt with a base and the like can be mentioned, and it is preferable to select a pharmacologically acceptable salt.

As the salts of glutamine, serine, histidine, arginine, valine, leucine, isoleucine, alanine and praline, salts similar to those described for cystine can be mentioned.

For the object of the present invention, the above-mentioned amino acids in a free form, and hydrochloride, acetate, malate, α-ketoglutarate, aspartate, glutamate, pyroglutamate thereof are preferable, and the above-mentioned amino acid in a free form, and hydrochloride thereof are more preferable.

In the present invention, glutamine, serine, histidine, arginine, valine, leucine, isoleucine, alanine and proline used in a free form or in the form of a salt may be extracted and purified from animals and plants etc. that are naturally present or obtained by a chemical synthesis method, a fermentation method, enzyme method or a gene recombination method. Commercially available products provided by each company may also be utilized.

When the agent of the present invention contains, as other amino acid in addition to cystine, at least one amino acid selected from the group consisting of glutamine, serine, histidine, arginine, valine, leucine, isoleucine, alanine and proline, the content of the amino acid is preferably not less than 0.1 wt %, more preferably not less than 1 wt %, particularly preferably not less than 3 wt %, relative to the total amino acid content of the agent of the present invention, from the aspects of preparation stability, function to improve physical capacity for activity, ease of taking and the like. The content of the amino acid is preferably not more than 99 wt %, more preferably not more than 90 wt %, particularly preferably not more than 80 wt %, relative to the total amino acid content of the agent of the present invention.

As used herein, when one amino acid selected from the aforementioned group is contained, the content of other amino acid means the content of the amino acid, and when two or more kinds of amino acids selected from the aforementioned group are contained, it means the total content thereof.

When the agent of the present invention contains, as other amino acid in addition to cystine, at least one amino acid selected from the group consisting of glutamine, serine, histidine, arginine, valine, leucine, isoleucine, alanine and proline, the content of the amino acid is generally not less than 5 mg, preferably not less than 10 mg, more preferably not less than 50 mg, particularly preferably not less than 100 mg as an intake or dose per one time in human, from the aspects of preparation stability, function to improve physical capacity for activity, ease of taking and the like. The content of the amino acid is preferably not more than 10 g, more preferably not more than 5 g, as an intake or dose per one time in human.

When the agent of the present invention contains, as other amino acid in addition to cystine, at least one amino acid selected from the group consisting of glutamine, serine, histidine, arginine, valine, leucine, isoleucine, alanine and proline, the total of the content of the amino acid and the content of cystine is preferably not less than 1.1 wt %, more preferably not less than 2 wt %, further preferably not less than 4 wt %, particularly preferably not less than 11 wt %, relative to the total amino acid content of the agent of the present invention, from the aspect of the improving effect on the physical capacity for activity. The total of the content of the amino acid and the content of cystine is preferably not more than 95 wt %, more preferably not more than 90 wt %, relative to the total amino acid content of the agent of the present invention.

When the agent of the present invention contains, as other amino acid in addition to cystine, at least one amino acid selected from the group consisting of glutamine, serine, histidine, arginine, valine, leucine, isoleucine, alanine and proline, the weight ratio of the content of cystine (a) and the content of at least one amino acid selected from the group consisting of glutamine, serine, histidine, arginine, valine, leucine, isoleucine, alanine and proline (b) ((a):(b)) is preferably 1:0.01 to 100, more preferably 1:0.01 to 50, further preferably 1:0.1 to 50, still more preferably 1:0.1 to 30, most preferably 1:1 to 25, from the aspect of the effect of each amino acid on improving physical capacity for activity.

When the agent of the present invention contains, as other amino acid in addition to cystine, at least one amino acid selected from the group consisting of isoleucine, leucine and valine, the weight ratio of the content of cystine (a) and the content of at least one amino acid selected from the group consisting of isoleucine, leucine and valine (b1) ((a):(b1)) is preferably 1:0.01 to 100, more preferably 1:0.01 to 50, further preferably 1:0.01 to 25, still more preferably 1:0.1 to 10.

When the agent of the present invention contains isoleucine, leucine and valine as other amino acids in addition to cystine, the weight ratio of each content thereof (isoleucine:leucine:valine) is generally 1:1.5 to 2.5:0.8 to 1.7, preferably 1:1.9 to 2.2:1.1 to 1.3. In addition, the weight ratio of the content of cystine (a) and the total content of isoleucine, leucine and valine (b2) ((a):(b2)) is preferably 1:0.01 to 100, more preferably 1:0.01 to 50, further preferably 1:0.1 to 25, still more preferably 1:0.5 to 20.

When the agent of the present invention contains glutamine as other amino acid in addition to cystine, the weight ratio of the content of cystine (a) and the content of glutamine (b3) ((a):(b3)) is preferably 1:0.01 to 100, more preferably 1:0.01 to 50, further preferably 1:0.01 to 25, still more preferably 1:0.1 to 10.

When the agent of the present invention contains alanine, or alanine and proline as other amino acid(s) in addition to cystine, the weight ratio of the content of cystine (a) and the content of alanine, or alanine and proline (b4) ((a):(b4)) is preferably 1:0.01 to 100, more preferably 1:0.01 to 50, further preferably 1:0.1 to 50, still more preferably 1:1 to 50, particularly preferably 1:5 to 25.

When the agent of the present invention contains proline as other amino acid in addition to cystine, the weight ratio of the content of cystine (a) and the content of proline (b5) ((a):(b5)) is preferably 1:0.01 to 100, more preferably 1:0.01 to 50, further preferably 1:0.01 to 25, still more preferably 1:0.1 to 10, particularly preferably 1:0.5 to 5.

The agent of the present invention may further contain amino acid(s) other than glutamine, serine, histidine, arginine, valine, leucine, isoleucine, alanine and proline, for example, essential amino acids such as threonine, methionine, phenylalanine and the like, non-essential amino acids such as aspartic acid, glycine, tyrosine and the like, and the like. It is preferable that the agent of the present invention does not substantially contain non-proteinogenic amino acid. When the agent of the present invention does not substantially contain non-proteinogenic amino acid, it is possible to sufficiently supply the protein starting material necessary for exercise and achieve function to improve physical capacity for activity even when the dose of the agent of the present invention is limited.

Examples of the non-proteinogenic amino acid include theanine, ornithine, citrulline, taurine, GABA (γ-amino butyric acid) and the like. Of these, since theanine cannot directly be a protein starting material, the agent of the present invention preferably does not substantially contain theanine.

In the present invention, non-proteinogenic amino acid (e.g., theanine etc.) is “not substantially contained” means (1) non-proteinogenic amino acid is not contained at all, or (2) non-proteinogenic amino acid is contained in an amount that does not affect the effect of the present invention (specifically, generally not more than 28 wt %, preferably not more than 20 wt %, more preferably not more than 10 wt %, further preferably not more than 1 wt %, particularly preferably not more than 0.1 wt %, relative to the content of all amino acids in the agent of the present invention).

The agent of the present invention can also be used in combination with other nutritional supplements and anti-fatigue agent and the like (hereinafter sometimes to be referred to as a concomitant drug). As the concomitant drug, carbohydrate preparation such as glucose, dextran and the like, fat emulsion such as purified soybean oil, purified egg-yolk lecithin and the like, protein preparation such as casein, whey protein and the like, caffeine, vitamins, minerals, polyphenols and the like can be specifically mentioned.

The agent of the present invention can also be used in combination with good bacteria such as lactic acid bacterium and bifidobacteria. Good bacteria are not particularly limited, and specifically, VSL#3 (registered trade mark) (InKine Pharmaceutical and Sigma-Tau Pharmaceuticals) and the like can be mentioned.

The agent of the present invention can have a dosage form of oral preparation such as tablet, coating tablet, chewable tablet, pill, (micro)capsule, granule, fine granule, powder, elixir, lemonade, syrup, suspension, emulsion, oral jelly and the like, injection such as solution, suspension, emulsion and the like, solid injection to be used by dissolving or suspending when in use, injectable preparation such as transfusion, sustainable injection and the like, and the like.

The agent of the present invention in the above-mentioned dosage form can be prepared by a formulating means well known in the field of preparations, for example, the methods described in the Japanese Pharmacopoeia preparation, sixteenth Edition General Rules [2] preparation, each article, the Japanese Pharmacopoeia preparation, seventeenth Edition, General Rules [3] preparation, each article, which are incorporated herein by reference in their entireties, and the like, and the like. In this case, various pharmacologically acceptable additives for preparations can be blended as necessary. The additive can be appropriately selected according to the dosage form of the agent of the present invention. For example, excipient, binder, disintegrant, lubricant, coating agent, base, solvent, diluent, solubilizing agent, solubilizer, emulsifier, dispersing agent, suspending agent, stabilizer, thickener, soothing agent, isotonicity agent, pH adjuster, antioxidant, antiseptic, preservative, corrigent, flavoring agent, sweetening agent, flavor, colorant and the like can be mentioned.

Specifically, examples of the excipient include magnesium carbonate, titanium dioxide, saccharides (e.g., lactose etc.), sugar alcohol (e.g., mannitol etc.), casein and the like.

Examples of the binder include gelatin, starch, cellulose and a derivative thereof and the like.

Examples of the disintegrant include crospovidone, crystalline cellulose and the like.

Examples of the lubricant include talc, magnesium stearate and the like.

Examples of the coating agent include methylmethacrylate. butylmethacrylate.dimethylaminoethylmethacrylate copolymer, ethylacrylate.methylmethacrylate. trimethylammmonioethylmethacrylate chloride copolymer and the like.

Examples of the base include animal and vegetable oil, polyethylene glycol and the like.

Examples of the solvent include purified water, water for injection, monovalent or polyhydric alcohol (e.g., glycerol etc.).

Examples of the emulsifier or dispersing agent include sorbitan fatty acid ester, glycerine fatty acid ester, polyoxyethylene sorbitan fatty acid ester, sucrose fatty acid ester and the like.

Examples of the stabilizer include adipic acid, β-cyclodextrin and the like.

Examples of the thickener include water-soluble polymer (e.g., sodium polyacrylate, carboxyvinyl polymer etc.), polysaccharides (sodium alginate, xanthan gum, tragacanth etc.) and the like.

Examples of the soothing agent include ethyl aminobenzoate, chlorobutanol, propylene glycol, benzyl alcohol and the like.

Examples of the isotonicity agent include potassium chloride, sodium chloride, sorbitol, physiological saline and the like.

Examples of the pH adjuster include hydrochloric acid, sulfuric acid, acetic acid, citric acid, lactic acid, sodium hydroxide, potassium hydroxide and the like.

Examples of the antioxidant include dibutylhydroxytoluene (BHT), butylhydroxyanisole (BHA), α-tocopherol, erythorbic acid and the like.

Examples of the antiseptic, or preservative include paraben (e.g., methylparaben etc.), benzyl alcohol, sodium dehydroacetate, sorbic acid and the like.

Examples of the corrigent or flavoring agent include ascorbic acid, erythritol, sodium L-glutamate and the like.

Examples of the sweetening agent include aspartame, licorice extract, saccharin and the like.

Examples of the flavor include l-menthol, d-camphor, cineol and the like.

Examples of the colorant include tar pigment (e.g., red No. 2, blue No. 1, yellow No. 4 etc.), inorganic pigment (e.g., red iron oxide, yellow iron oxide, black iron oxide etc.), natural dye (e.g., annatto dye, turmeric dye, β-carotene etc.) and the like.

The ingestion amount or dose of the agent of the present invention is appropriately determined according to the condition or symptom, sex, age of the target to be applied to (hereinafter to be referred to as the “application target” in the present specification), dosage form of the agent of the present invention, administration method and the like. When the application target is a human adult, the agent of the present invention can be used for the ingestion or administration of generally 0.1 mg/kg body weight to 2 g/kg body weight, preferably 1 mg/kg body weight to 1 g/kg body weight, more preferably 5 mg/kg body weight to 0.2 g/kg body weight, of cystine per day.

When the agent of the present invention contains, in addition to cystine, at least one amino acid selected from the group consisting of glutamine, serine, histidine, arginine, valine, leucine, isoleucine, alanine and proline as other amino acid, and the application target is an adult, the agent of the present invention can be used for the ingestion or administration of at least one amino acid selected from the group consisting of glutamine, serine, histidine, arginine, valine, leucine, isoleucine, alanine and proline in the following amount.

glutamine: generally 0.1 mg/kg body weight to 5 g/kg body weight, preferably 1 mg/kg body weight to 4 g/kg body weight, more preferably 5 mg/kg body weight to 2 g/kg body weight, per day

serine: generally 0.1 mg/kg body weight to 4 g/kg body weight, preferably 1 mg/kg body weight to 2 g/kg body weight, more preferably 5 mg/kg body weight to 1 g/kg body weight, per day

histidine: generally 0.1 mg/kg body weight to 4 g/kg body weight, preferably 1 mg/kg body weight to 2 g/kg body weight, more preferably 5 mg/kg body weight to 1 g/kg body weight, per day

arginine: generally 1 mg/kg body weight to 4 g/kg body weight, preferably 1 mg/kg body weight to 2 g/kg body weight, more preferably 20 mg/kg body weight to 1 g/kg body weight, per day

valine: generally 0.1 mg/kg body weight to 4 g/kg body weight, preferably 1 mg/kg body weight to 2 g/kg body weight, more preferably 5 mg/kg body weight to 1 g/kg body weight, per day

leucine: generally 0.1 mg/kg body weight to 4 g/kg body weight, preferably 1 mg/kg body weight to 2 g/kg body weight, more preferably 5 mg/kg body weight to 1 g/kg body weight, per day

isoleucine: generally 1 mg/kg body weight to 4 g/kg body weight, preferably 1 mg/kg body weight to 2 g/kg body weight, more preferably 5 mg/kg body weight to 1 g/kg body weight, per day

alanine: generally 0.1 mg/kg body weight to 4 g/kg body weight, preferably 1 mg/kg body weight to 2 g/kg body weight, more preferably 5 mg/kg body weight to 1 g/kg body weight, per day

proline: generally 0.1 mg/kg body weight to 4 g/kg body weight, preferably 1 mg/kg body weight to 2 g/kg body weight, more preferably 5 mg/kg body weight to 1 g/kg body weight, per day

The above-mentioned amount of ingestion or dose can be taken once or in two or more portions (e.g., 2 to 5 portions) per day.

When the agent of the present invention is applied to a human adult, the agent of the present invention can be used such that the total ingestion amount or dose of all amino acids would be generally 0.1 mg/kg body weight to 5 g/kg body weight per day. Particularly, when the application target is, for example, a person who particularly desires to improve physical capacity for activity (e.g., muscular endurance capacity, whole body endurance capacity and the like) such as athlete or the like, the agent of the present invention can be used such that the total ingestion amount or dose of all amino acids would be preferably 10 mg/kg body weight to 2 g/kg body weight, more preferably 50 mg/kg body weight to 1 g/kg body weight, per day, from the aspects of the function of the agent of the present invention to improve physical capacity for activity and the protein amount to be ingested by an athlete and the like per day.

The timing of ingestion or administration of the agent of the present invention is not particularly limited, and may be, for example, before start of exercise, during exercise, after completion of the exercise or the like.

While the number of ingestion or administration of the agent of the present invention is not particularly limited, it is at least once (once or twice or more) when the physical capacity for activity needs to be improved. From the viewpoint of convenience of application, the agent of the present invention is preferably ingested or administered at least once before starting exercise or during the exercise or after completion of the exercise. Also, the agent of the present invention may be ingested or administered at least once before start of exercise, and further at least once during exercise and/or after completion of the exercise.

When the number of ingestions or administrations of the agent of the present invention is 2 or more, while the ingestion or administration period (period from the first ingestion or administration to the last ingestion or administration) of the agent of the present invention is not particularly limited, it is generally 6 hr to 4 weeks. To exhibit the effect more, it is preferably 1 day to 2 weeks, more preferably 3 days to 1 week.

In one embodiment, the agent of the present invention is ingested or administered 1 to 5 times (preferably 1 to 3 times) per day from one day to 2 weeks before (preferably 3 days to 1 week before) starting exercise. When the agent of the present invention is ingested after completion of the exercise, the agent can be ingested or administered 1 to 3 times immediately after completion of exercise to after 24 hr (preferably immediately after to after 6 hr).

When the agent of the present invention contains cystine and at least one amino acid selected from the group consisting of glutamine, serine, histidine, arginine, valine, leucine, isoleucine, alanine and proline, these may be contained singly or in any combination in two or more preparations, or all may be contained in a single preparation. When these active ingredients are contained in two or more preparations, the timing of ingestion or administration of each preparation may be simultaneous or separately, and also, the pathways of ingestion or administration of respective preparations may be the same or different. An embodiment containing all active ingredients in one preparation is preferable for the agent of the present invention, since it can be ingested or administered conveniently. When the above-mentioned concomitant drug is used in addition to the amino acid such as cystine, glutamic acid and the like, the method for ingestion or administration thereof (other preparation or the same preparation) and the timing of ingestion or administration thereof can be appropriately determined depending on the kind and effect of the concomitant drug.

In the present invention, when cystine and at least one selected from the group consisting of glutamine, serine, histidine, arginine, valine, leucine, isoleucine, alanine, and proline are contained in two or more preparations, the content of each of these (content per one intake or dose, and content relative to the content of all amino acids in the agent of the present invention) and weight ratio are shown by totaling the amounts contained in each preparation.

The agent of the present invention can be formulated as a unit package form. In the present specification, the “unit package form” means a form of one or more units with a particular amount (e.g., intake per one time etc.) as one unit is/are packed in one container or package. For example, a unit package form with intake or dose per one time as one unit is referred to as “unit package form for intake or dose per one time”. A container or package used for the unit package form can be appropriately selected according to the form and the like of the agent of the present invention. For example, paper container or bag, plastic container or bag, pouch, aluminum can, steel can, glass bottle, pet bottle, PTP (press through pack) package sheet and the like can be mentioned.

The application target of the agent of the present invention includes, for example, mammals (e.g., human, mouse, rat, hamster, rabbit, cat, dog, bovine, horse, donkey, swine, sheep, monkey etc.) and birds (e.g., chicken etc.). When the agent of the present invention is applied to an application target animal other than human (hereinafter to be also simply referred to as “target animal”), the ingestion amount or dose of the agent of the present invention can be appropriately set according to the kind, sex, body weight and the like of the target animal.

The agent of the present invention can improve physical capacity for activity, particularly, the ability to continue performance like muscular endurance capacity and whole body endurance capacity.

Particularly, the agent of the present invention enhances expression of glucose transporter genes such as glucose transporter 4 (GLUT4) and the like present in the adipose tissue and striated muscle (cardiac muscle and skeletal muscle) and involved in the glucose uptake, and enhances the uptake of glucose to be utilized as an energy source. Therefore, the agent can effectively improve physical capacity for activity.

In addition, the agent of the present invention can increase the amount of glycogen in muscle. Therefore, the agent of the present invention can be used as an agent for increasing the amount of glycogen in muscle. Particularly, it is also preferably used as a glycogen loading promoter that stores glycogen in the body, and the can increase the limit of exercise capacity.

Furthermore, the agent of the present invention can further enhance the effects of exercise (training) such as increase in the amount of glycogen in muscle, improved muscular endurance capacity, improved whole body endurance capacity and the like.

The agent of the present invention can be used as it is or added with the above-mentioned additives such as excipient, solvent, diluent and the like to give a medicament to be administered to athlete who is required to improve physical capacity for activity, elderly people, person in need of nursing care, patients under exercise therapy and the like.

Furthermore, the agent of the present invention can be used by adding to various foods. The food to which the agent of the present invention is added is not particularly limited, and may be any as long as it is a food in the form generally served for meals. For example, the agent of the present invention is added to drinks, and a suitable flavor is added when desired, whereby a drink (e.g., beverage etc.) can be provided. More specifically, the agent of the present invention can be added to, for example, juice, milk, confectionery, jelly, yogurt, candy and the like.

The present invention also provides a food composition for improving physical capacity for activity containing the agent of the present invention (hereinafter to be also referred to as “the food composition of the present invention”).

The food composition of the present invention contains the agent of the present invention and, where necessary, food additives such as production agent, thickening stabilizer, gum base, emulsifier, preservation, antioxidant, gloss agent, pH adjuster, sweetener, bitter taste, acidulant, colorant, flavor and the like. Alternatively, the food composition can be provided in various forms containing the agent of the present invention and food or food starting materials, for example, drinks such as juice, beverage water, teas and the like; milk products such as lactobacillus drinks, fermented milk, butter, cheese, yogurt, processing milk, defatted milk and the like; meat products such as ham, sausage, hamburg steak and the like; fish meat paste products such as boiled fish paste, tube-like fish sausage, satsuma-age and the like; egg products such as rolled Japanese-style omelette, egg tofu and the like; confectionerys such as cookie, jelly, chewing gum, candy, snack confectionery, frozen dessert and the like; bread; noodles; pickles; smoked product; dried fish; food boiled down in soy; salt-preserved product; soups; seasonings, bottled food, canned food, retort pouch food. In addition, forms such as powder, granule, sheet, capsule, tablet, jelly and the like can be provided.

The food composition of the present invention can be preferably ingested by athlete who is required to improve physical capacity for activity, elderly people, person in need of nursing care, patients under exercise therapy and the like.

The food composition of the present invention can be preferably ingested widely by those who desire to improve physical capacity for activity such as those who are not top-elite athletes but routinely exercise well, middle or advanced-aged people who are not elderly but desire to maintain or improve physical capacity for activity, those who do not need nursing care but require rehabilitation due to injury, disease and the like.

Therefore, the food composition of the present invention can also be provided as food with health claims such as food for specified health uses, food with nutrient function claims, indicated functional food and the like for the maintenance or improvement of physical capacity for activity, special purpose foods such as food for sick people, food for the elderly and the like, health supplement and the like.

Furthermore, the agent of the present invention can be used by adding to a high density liquid diet or food supplement.

The “high density liquid diet” is a comprehensive nutritional food (liquid diet) adjusted to a concentration of about 1 kcal/mL, which is designed based on the daily nutritional requirement and with sufficient consideration of the qualitative composition of each nutrient so that remarkable excessive or insignificant nutrients will not occur even when only this is ingested for a long period of time.

The “food supplement” in the present invention refers to one ingested to aid nutrition other than one ingested as a food, and also includes nutritional supplement, supplement and the like. When the agent of the present invention is added to a dietary supplement, it can be prepared in a form such as tablet, capsule, powder, granule, suspension, chewable, syrup and the like by adding other nutrition components and additives when desired.

The above-mentioned food composition of the present invention can be processed and produced by adding food additive as necessary to the agent of the present invention or adding the agent of the present invention to a food or food starting materials, and applying a general food production method.

The content of cystine, and the content of at least one amino acid selected from the group consisting of glutamine, serine, histidine, arginine, valine, leucine, isoleucine, alanine and proline in the food composition of the present invention are appropriately determined according to the kind or form of the food composition, the level of the improving effect on the physical capacity for activity expected by the ingestion of the food composition and the like. The content of cystine is generally about 1 wt % to 95 wt %, preferably about 3 wt % to 90 wt %, more preferably about 10 wt % to 80 wt %, relative to the total weight of the food, and is about 1 wt % to 95 wt %, preferably about 3 wt % to 90 wt %, relative to the total weight of at least one amino acid selected from the group consisting of glutamine, serine, histidine, arginine, valine, leucine, isoleucine, alanine and proline.

The ingestion amount of the food composition of the present invention can be set as an amount permitting ingestion of the aforementioned amount of cystine and the like in the agent of the present invention.

The agent of the present invention may also be provided in the embodiment of a commercial package containing a written matter stating that the agent can or should be used for improving physical capacity for activity.

Furthermore, the present invention also provides a method for improving physical capacity for activity of a target animal in need of improvement of the physical capacity for activity (hereinafter to be also referred to as “the method of the present invention” in the present specification).

The method of the present invention comprises ingestion or administration of cystine in an amount effective for improving physical capacity for activity of a target animal in need of improvement of the physical capacity for activity.

The method of the present invention further comprises ingestion or administration of, in addition to cystine, at least one amino acid selected from the group consisting of glutamine, serine, histidine, arginine, valine, leucine, isoleucine, alanine and proline in an amount effective for improving physical capacity for activity of a target animal in need of improvement of the physical capacity for activity.

As the target animal of the method of the present invention, human and mammals other than human such as mouse, rat, hamster, rabbit, cat, dog, bovine, horse, donkey, swine, sheep, monkey and the like, and birds such as chicken and the like can be mentioned.

When the target animal of the method of the present invention is human, the method of the present invention can be widely applied to those who wish to improve their physical capacity for activity such as muscular endurance capacity, whole body endurance capacity, and the like, including those who exercise and those with a declining tendency of physical functions, exercise capacity and the like. Particularly, it can be preferably applied to athlete who is desired to improve physical capacity for activity, elderly people and person in need of nursing care, each with a declining tendency of the ability to continue performance such as muscular endurance capacity and whole body endurance capacity, patients having a disease such as diabetes and the like and under exercise therapy and the like.

While the ingestion amount or dose of cystine and the like in the method of the present invention is determined according to the kind, age, symptom, condition and the like of the target animal, an amount similar to the above-mentioned ingestion amount or dose of the agent of the present invention for a human or a target animal other than human can be ingested or administered at the frequency and period mentioned above.

In the method of the present invention, the weight ratio of the ingestion amount or dose per one time of cystine (c) and the ingestion amount or dose per one time of at least one amino acid selected from the group consisting of glutamine, serine, histidine, arginine, valine, leucine, isoleucine, alanine and proline (d) ((c):(d)) is preferably 1:0.001 to 100, more m preferably 1:0.01 to 100.

The ingestion or administration method of cystine and the like in the method of the present invention includes oral administration, enteral tube administration, administration by infusion and the like. Oral administration is preferable since convenient ingestion is possible without the need to perform under the guidance and supervision of a doctor at a medical institution.

Other features of the invention will become apparent in the course of the following descriptions of exemplary embodiments which are given for illustration of the invention and are not intended to be limiting thereof.

### EXAMPLES

### Experimental Example 1. Study of Endurance-Improving Effect of Cystine

Athletes repeat strenuous exercise and then improve physical functions. Therefore, the effect of cystine intake on the improvement of endurance capacity was examined by the following test.

6-Week-old male CD2F1 mice (CHARLES RIVER LABORATORIES JAPAN, INC.) were divided into 3 groups of a group with ingestion of a normal diet (AIN-93G composition) and without exercise (non-exercise group), a group with ingestion of a normal diet and with exercise (exercise group), and a group with ingestion of a diet containing 2 wt % cystine (free form), which was prepared by replacing casein in the normal diet with cystine, and with exercise (exercise+2% cystine group). Each group ingested a test diet (normal diet or normal diet containing 2 wt % cystine) for 8 days. Thereafter, the exercise group and the exercise+2% cystine group were made to run on a treadmill until exhaustion for 3 consecutive days, and the running time until then was measured (FIG. 1). The non-exercise group was fasted and deprived of water during running of the other two groups. On the next day after completion of the 3-day running, the muscle of the mice in each group was collected, and the amount of glycogen in the muscle was measured.

Running was performed at running speed=18 m/min for 30 min using a treadmill for small animal, manufactured by ARCO system inc. Thereafter, the running speed was increased by 3 m/min every 30 min and after reaching the running speed of 36 m/min, running speed was kept as 36 m/min. When the mouse dropped to the back of the lane and could not return to the traveling lane and restart the running even when tapped, the mouse was judged to have been exhausted.

The amount of glycogen in muscle was measured as follows. The gastrocnemius muscle (50 mg) of mouse was placed in 300 μL of 30 wt % potassium hydroxide solution, and digested for 30 min on a heat block (100° C.). Glycogen was extracted and the digestion solution was neutralized with saturated aqueous sodium sulfate solution. Thereafter, 95 vol % ethanol was added, and the mixture was centrifuged to form pellets. This operation was repeated twice, and 0.6 N hydrochloric acid was added to the obtained precipitate. The mixture was treated for 2 hr on a heat block for hydrolysis to give glucose. Glucose in this solution was detected and quantified using “glucose CII-Test Wako” (manufactured by Wako Pure Chemical Industries, Ltd.). The obtained glucose amount was multiplied by 0.93 to calculate the amount of glycogen in the muscle.

The running time until exhaustion and the amount of glycogen in gastrocnemius muscle were tested by analysis of variance and followed by the Tukey test.

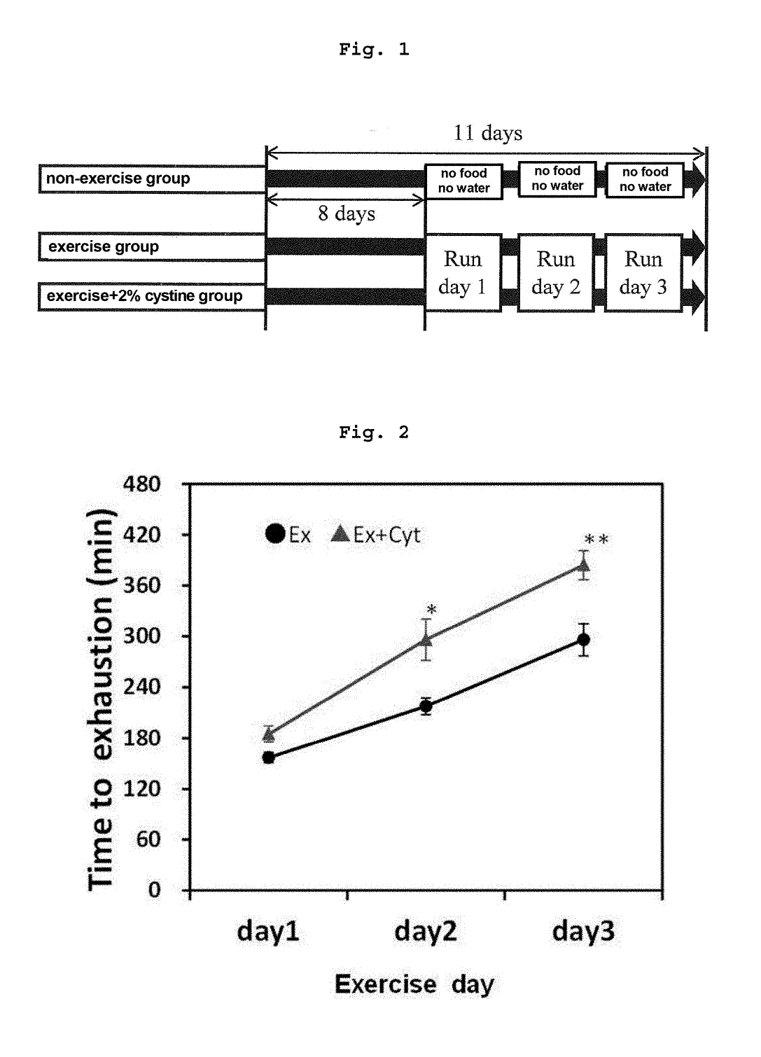

The running time until exhaustion on day 1, day 2 and day 3 of exercise are shown in FIG. 2. In both groups (exercise group (Ex) and exercise+2% cystine group (Ex+Cyt)), the running time until exhaustion increased by doing exercise every day. Thus, it was shown that exercise improves endurance capacity. The amount of increase in the running time by exercise was significantly (P<0.05 or P<0.01) higher in the exercise+2% cystine group (Ex+Cyt) than in the exercise group (Ex). The results have clarified that cystine potentiates the endurance-improving effect of exercise.

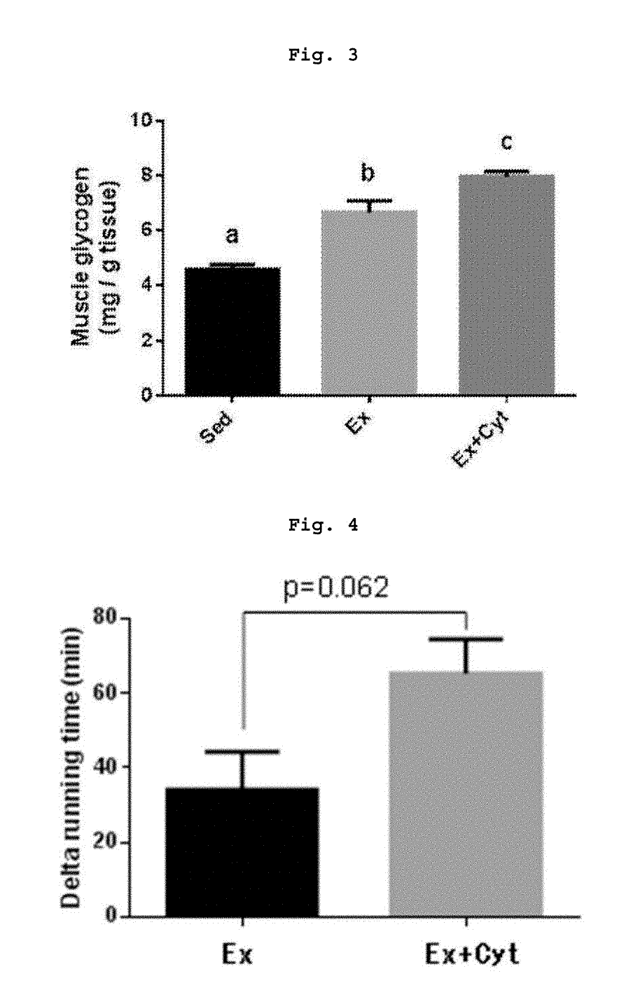

The amount of glycogen in gastrocnemius muscle was measured the next day of 3-day continuous exercise. The results are shown in FIG. 3. The amount of glycogen in gastrocnemius muscle in the exercise group (Ex) after continuous exercise for 3 days significantly (P<0.05) increased compared to the non-exercise group (Sed). In the exercise+2% cystine group (Ex+Cyt), the amount of glycogen in gastrocnemius muscle further significantly (P<0.05) increased than in the exercise group (Ex).

From the above results, it was concluded that cystine further increases the amount of glycogen in muscle efficiently than that by simple exercise.

### Experimental Example 2. Study of Influence of Timing of Cystine Ingestion on Endurance-Improving Effect

In Experimental Example 1, an endurance-improving effect of cystine was found by ingestion of cystine for 8 days before exercise. It is also investigated whether cystine ingestion after the exercise can improve endurance capacity.

6-Week-old male CD2F1 mice (CHARLES RIVER LABORATORIES JAPAN, INC.) were given a normal diet (AIN-93G composition) for 8 days. Thereafter, the mice were forced to run until exhaustion under the exercise conditions similar to those in Experimental Example 1, and the running time then was measured. After exercise, the aforementioned mice were divided into 2 groups of a group with normal diet ingestion (exercise group) and a group with a diet containing 2 wt % cystine (free form), which was prepared by replacing casein in the normal diet with cystine (exercise+2% cystine group). Each group ingested a test diet (normal diet or normal diet containing 2 wt % cystine). The next day, the mice were made to run until exhaustion under the exercise conditions similar to those in Experimental Example 1, and the running time then was measured. The amount of increase in the running time was calculated as an endurance-improving effect. As for the calculation results of the amount of increase in the running time, unpaired t-test was performed.

The amount of increase in the running time is shown in FIG. 4. The amount of increase in the running time tended to be higher in the exercise+2% cystine group (Ex+Cyt) than in the exercise group (Ex). The results suggest that cystine potentiates the endurance-improving effect by ingestion of cystine for one day after exercise.

### Example 1. Agent for Improving Physical Capacity for Activity

A composition obtained by mixing each amino acid (hydrochloride was used for lysin free form was used for other amino acids) according to the composition shown in Table 1 was taken as the agent for improving physical capacity for activity of Example 1. The amino acid composition reported in patent document 2 to have an endurance-improving effect was used as Comparative Example and the composition is also shown in Table 1.

TABLE 1

| content (wt %) | Comparative Example | Example 1 |
|---|---|---|
| Asp | 6.5 | 2.2 |
| Thr | 4.3 | 1.4 |
| Ser | 5.5 | 1.8 |
| Glu | 20.3 | 6.8 |
| Pro | 10.6 | 3.5 |
| Gly | 1.8 | 0.6 |
| Ala | 2.8 | 0.9 |
| Val | 6.2 | 2.1 |
| Met | 2.6 | 0.9 |
| Ile | 5.0 | 1.7 |
| Leu | 8.8 | 2.9 |
| Tyr | 5.4 | 1.8 |
| Phe | 4.8 | 1.6 |
| Lys | 7.5 | 2.5 |
| Trp | 1.5 | 0.5 |
| His | 2.7 | 0.9 |
| Arg | 3.4 | 1.1 |
| Cyt | 0.3 | 66.8 |
| total | 100 | 100 |

### Experimental Example 3. Study of Endurance-Improving Effect of the Agent for Improving Physical Capacity for Activity of Example 1

An endurance-improving effect has been reported for the amino acid composition containing cystine disclosed in patent document 2 (amino acid composition of Comparative Example). Therefore, whether the cystine content of the amino acid compositions can affect the endurance-improving effect was studied by the following test.

6-Week-old male CD2F1 mice (CHARLES RIVER LABORATORIES JAPAN, INC.) were divided into 4 groups of groups with ingestion of a normal diet containing 3 wt % amino acid composition of Comparative Example (Table 1) (AIN-93G composition) and without exercise (Sed-Con) or with exercise (Ex-Con), and groups with ingestion of a normal diet containing 3 wt % of the agent for improving physical capacity for activity of Example 1 (Table 1) and without exercise (Sed-Cyt) or with exercise (Ex-Cyt). A test diet (normal diet containing the amino acid composition of Comparative Example or a normal diet containing the agent for improving physical capacity for activity of Example 1) was given for 7 days. The groups with exercise were forced to run in a treadmill until exhaustion for 2 consecutive days and the running time then was measured. The non-exercise groups were fasted and deprived of water during running of the exercise groups. On the next day after completion of the 2-day running, the gastrocnemius muscle of the mice in each group was collected, and the amount of glycogen in the muscle was measured similarly to Experimental Example 1. The running protocol was identical to the running protocol in Experimental Example 1.

The measurement results of the running time until exhaustion and the amount of glycogen in gastrocnemius muscle were tested by analysis of variance followed by the Tukey test. As for the detection of the amount of increase in the running time, unpaired t-test was used.

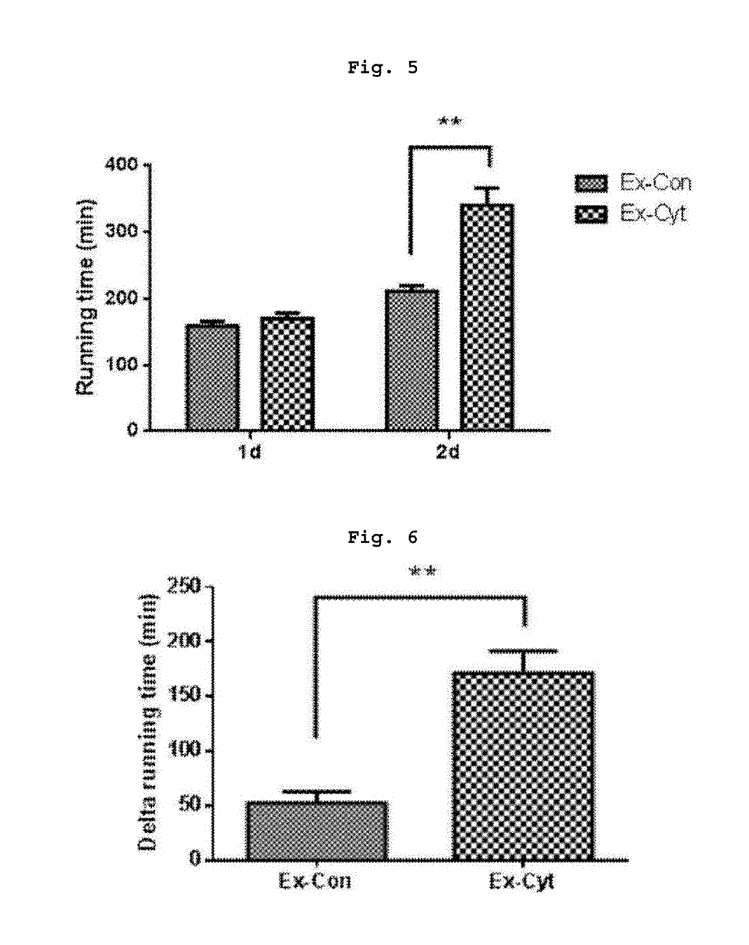

The running time until exhaustion on day 1 (1d) and day 2 (2d) of exercise are shown in FIG. 5.

In addition, the difference (increase) in the running time between day 1 and day 2 of exercise is shown in FIG. 6. The running time of 1d did not show difference between the exercised groups. As to the running time of 2d, the group with ingestion of a noLlual diet containing the agent for improving physical capacity for activity of Example 1 and with exercise (Ex-Cyt) showed a significant (P<0.01) increase in the running time until exhaustion compared to the group with ingestion of a normal diet containing the amino acid composition of Comparative Example and with exercise (Ex-Con). As to the difference in the running time between day 1 and day 2, Ex-Cyt showed a significantly (P<0.01) high value compared to Ex-Con.

From the above results, it was concluded that the agent for improving physical capacity for activity of the present invention containing cystine at a higher content than the amino acid composition containing cystine disclosed in patent document 2 efficiently improves endurance capacity by exercise.

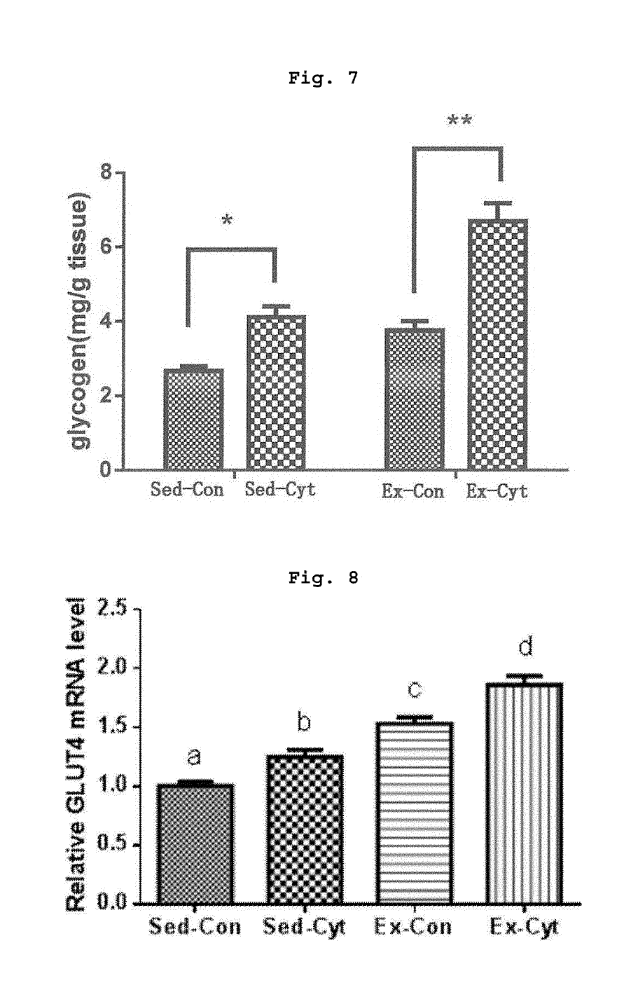

The amount of glycogen in gastrocnemius muscle in each JO group is shown in FIG. 7. The group with ingestion of a normal diet containing the agent for improving physical capacity for activity of Example 1 and without exercise (Sed-Cyt) showed a significantly (P<0.05) high value of the amount of glycogen in gastrocnemius muscle compared to the group with ingestion of a normal diet containing the amino acid composition of Comparative Example and without exercise (Sed-Con), and it was clarified that the agent for improving physical capacity for activity of the present invention containing cystine at a high content increases the amount of glycogen in muscle.

Furthermore, when exercise was continued for 2 days, the results of the group with ingestion of a normal diet containing the amino acid composition of Comparative Example and with exercise (Ex-Con) showed that the amount of glycogen in gastrocnemius muscle increases by exercise alone. The group with ingestion of a normal diet containing the agent for improving physical capacity for activity of the present invention containing cystine at a high content and with exercise (Ex-Cyt) showed a further increase in the amount of glycogen in gastrocnemius muscle (significant with P<0.01).

From the above results, it was found that mere ingestion of the agent for improving physical capacity for activity of Example 1 containing cystine at a high content increases the amount of glycogen in muscle as compared to the amino acid composition of Comparative Example having a low cystine content, and that exercise can further enhance the effect of increasing the amount of glycogen in muscle.

The expression of gene regarding glucose uptake in gastrocnemius muscle in each group was studied as follows.

Total RNA was extracted from the gastrocnemius muscle collected from the mice in each group by using RNeasy Fibrous Tissue Mini Kit (QIAGEN). The concentration of the obtained total RNA was measured using NanoDrop system (LMS Co., Ltd.), reverse transcription was performed using Prime Script RT reagent Kit (Takara Bio Inc.) and cDNA was obtained. The obtained cDNA was subjected to real-time PCR, the expression level of glucose transporter 4 (GLUT4) gene was evaluated (instrument used: Thermal Cycler Dice TP800 (Takara Bio Inc.), reagent: SYBR Premix Ex Taq (registered trade mark) II KiT (Takara Bio Inc.). As the primer sequences of GLUT4 gene, 5′-AATGGAGACTGATGCGCTCT-3′ (SEQ ID NO: 1) and 5′-ACTCTTGCCACACAGGCTCT-3′ (SEQ ID NO: 2) were used. As the housekeeping gene, Gapdh was used.

The expression level of the GLUT4 gene are shown by a value relative to the expression level of the group with ingestion of a diet containing the amino acid composition of Comparative Example and without exercise (Sed-Con) as 1. The detection of the statistically significant difference was tested by analysis of variance followed by the Tukey test after.

The expression level of the GLUT4 gene in gastrocnemius muscle in each group is shown in FIG. 8. The group with ingestion of a diet containing the agent for improving physical capacity for activity of Example 1 and without exercise (Sed-Cyt) showed a significantly (P<0.05) high value of the expression of GLUT4 in the gastrocnemius muscle compared to the group with ingestion of a diet containing the amino acid composition of Comparative Example and without exercise (Sed-Con). The results suggest that the agent for improving physical capacity for activity of the present invention containing cystine at a high content increases the expression level of GLUT4 gene in the muscle.

Also, in the group with ingestion of a normal diet containing the amino acid composition of Comparative Example and with exercise (Ex-Con), the expression level of GLUT4 gene was high as compared to Sed-Con, and it was shown that, when exercise was continuously performed, the expression level of GLUT4 gene in gastrocnemius muscle increases even by exercise alone. In the group with ingestion of a diet containing the agent for improving physical capacity for activity of Example 1 of the present invention containing cystine at a high content and with exercise (Ex-Cyt), the expression level of GLUT4 gene in gastrocnemius muscle is higher than that in Ex-Con (significant at P<0.05).

From the above results, it was clarified that ingestion of the agent for improving physical capacity for activity of the present invention containing cystine at a high content can improve physical capacity for activity by increasing the transporter that incorporates glucose into muscle. Furthermore, it was found that exercise and ingestion of the agent for improving physical capacity for activity of the present invention can further enhance the effect of improving physical capacity for activity.

### Example 2. Agent for Improving Physical Capacity for Activity

A composition obtained by mixing each amino acid (in free form) according to the composition shown in Table 2 was taken as the agent for improving physical capacity for activity in Example 2.

TABLE 2

| content (wt %) | Example 2 |
|---|---|
| glutamine | 81.25 |
| cystine | 18.75 |
| total | 100 |

### Experimental Example 4. Study of Endurance-Improving Effect of the Agent for Improving Physical Capacity for Activity of Example 2

The endurance-improving effect of the agent for improving physical capacity for activity of Example 2 was studied by the following test.

6-Week-old male CD2F1 mice (CHARLES RIVER LABORATORIES JAPAN, INC.) were divided into 2 groups of a group with JO ingestion of a normal diet (AIN-93G composition) and with exercise (Ex-Con), and a group with ingestion of a normal diet containing 3.2 wt % of the agent for improving physical capacity for activity of Example 2 (Table 2) and with exercise (Ex-CG). A test diet (normal diet or normal diet containing the agent for improving physical capacity for activity of Example 2) was given for 7 days. The groups were made to run in a treadmill until exhaustion for 2 consecutive days and the running time then was measured. On the next day after completion of the 2-day running, the gastrocnemius muscle of the mice in each group was collected, and the amount of glycogen in the muscle was measured similarly to Experimental Example 1. The running protocol was identical to the running protocol in Experimental Example 1.

The running time until exhaustion was tested by analysis of variance followed by the Tukey test in the same manner as in Experimental Example 1. As for the detection of the amount of increase in the running time and the amount of glycogen in gastrocnemius muscle, unpaired t-test was used.

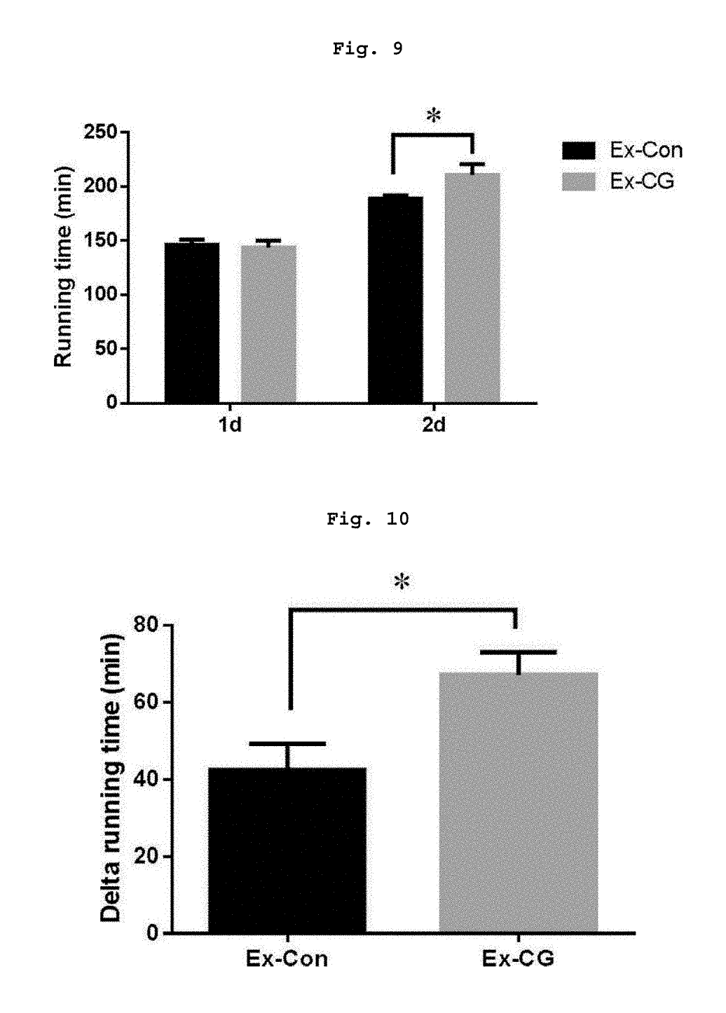

The running time until exhaustion on day 1 (1d) and day 2 (2d) of exercise are shown in FIG. 9. In addition, the difference (increase) in the running time between day 1 and day 2 of exercise is shown in FIG. 10. The running time of 1d did not show difference between the exercised groups. As to the running time of 2d, the group with ingestion of a normal diet containing the agent for improving physical capacity for activity of Example 2 and with exercise (Ex-CG) showed a significant (P<0.05) increase in the running time until exhaustion compared to the group with ingestion of a normal diet and with exercise (Ex-Con). As to the difference in the running time between day 1 and day 2, Ex-CG showed a significantly (P<0.05) high value compared to Ex-Con.

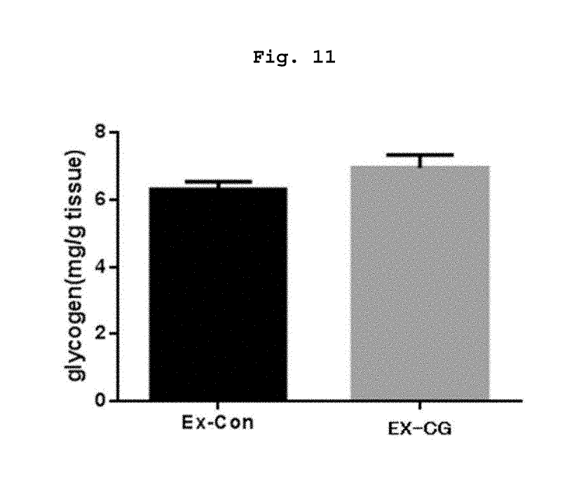

The amount of glycogen in gastrocnemius muscle in each group is shown in FIG. 11. In the group with ingestion of a normal diet containing the agent for improving physical capacity for activity of Example 2 and with exercise (Ex-CG), the amount of glycogen in gastrocnemius muscle is higher than that in the group with ingestion a normal diet and with exercise (Ex-Con).

From the above results, it was clarified that the agent for improving physical capacity for activity of Example 2 of the present invention containing cystine and glutamine efficiently improves endurance capacity by exercise, and has an effect to increase the amount of glycogen in muscle.

### INDUSTRIAL APPLICABILITY

As described in detail above, the present invention can provide an agent for improving physical capacity for activity that can be preferably used for improving physical capacity for activity, particularly, the ability to continue physical activity such as muscular endurance capacity and whole body endurance capacity. Especially, the agent for improving physical capacity for activity of the present invention increases the amount of glycogen in muscle and is preferably used as an agent for increasing the amount of glycogen in muscle, particularly a glycogen loading promoter. Furthermore, the agent for improving physical capacity for activity of the present invention can enhance the effect of increasing glucose uptake in muscle and the like by exercise (training), the effect of increasing the amount of glycogen in muscle, and the effect of improving muscular endurance capacity and whole body endurance capacity.

In addition, the agent for improving physical capacity for activity of the present invention contains amino acids rich in food experience such as cystine and the like as the active ingredient. Therefore, the agent has high safety, hardly causes side effects, and is extremely advantageous in the fields of pharmaceutical product, food and the like.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. An agent for improving physical capacity for activity, comprising cystine as an active ingredient.

2. The agent according to claim 1, wherein the agent improves an ability to continue physical activity.

3. The agent according to claim 1, wherein the content of cystine is 2.5 mg to 15 g as an intake or dose per one time.

4. The agent according to claim 1, wherein the content of cystine is not less than 1 wt % relative to the total amino acid content.

5. The agent according to claim 1, further comprising at least one amino acid selected from the group consisting of glutamine, serine, histidine, arginine, valine, leucine, isoleucine, alanine, and proline.

6. The agent according to claim 5, wherein a total of the content of cystine and the content of at least one amino acid selected from the group consisting of glutamine, serine, histidine, arginine, valine, leucine, isoleucine, alanine and proline is not less than 1.1 wt % relative to the total amino acid content.

7. The agent according to claim 5, wherein a weight ratio of the content of cystine (a) and the content of at least one amino acid selected from the group consisting of glutamine, serine, histidine, arginine, valine, leucine, isoleucine, alanine and proline (b) ((a):(b)) is 1:0.01 to 100.

8. The agent according to claim 1, wherein the agent is in a form suitable for oral ingestion or oral administration.

9. The agent according to claim 1, wherein the agent improves muscular endurance capacity or whole body endurance capacity.

10. The agent according to claim 1, wherein the agent increases the amount of glycogen in muscle.

11. The agent according to claim 10, wherein the agent is a glycogen loading promoter.

12. A medicament, comprising the agent according to claim 1.

13. A food composition, comprising the agent according to claim 1.

14. A method for improving physical capacity for activity, comprising ingestion by or administration to a subject in need thereof of cystine in an amount effective for improving physical capacity for activity to a subject animal in need of improvement of physical capacity for activity.

15. The method according to claim 14, wherein the method improves the ability to continue physical activity.

16. The method according to claim 14, comprising an intake or dose per one time of cystine of 2.5 mg to 15 g.

17. The method according to claim 14, further comprising ingestion or administration of at least one amino acid selected from the group consisting of glutamine, serine, histidine, arginine, valine, leucine, isoleucine, alanine, and proline in an amount effective for improving physical capacity for activity.

18. The method according to claim 17, comprising an intake or dose per one time of the at least one amino acid selected from the group consisting of glutamine, serine, histidine, arginine, valine, leucine, isoleucine, alanine and proline of 10 mg to 10 g.

19. The method according to claim 17, wherein a weight ratio of the intake or dose per one time of cystine (c) and the intake or dose per one time of at least one amino acid selected from the group consisting of glutamine, serine, histidine, arginine, valine, leucine, isoleucine, alanine and proline (d) ((c):(d)) is 1:0.01 to 100.

20. The method according to claim 14, comprising oral ingestion or oral administration of said cystine.

21. The method according to claim 14, comprising ingestion or administration of said cysteine at least once before start of exercise.

22. The method according to claim 14, comprising ingestion or administration of said cysteine at least once during exercise or after completion of exercise.
